(19) United States
(12) Patent Application Publication
(10) Pub. No.: US 2016/0151119 A1
(43) Pub. Date: Jun. 2, 2016

(54) SURGICAL GUIDANCE SYSTEM

(71) Applicant: Arthrex GmbH, Munchen (DE)

(72) Inventors: Gerlinde Michel, Munchen (DE); Jan Stifter, Schneisingen (CH); Michael Hofmann, Timmendorfer Strand (DE)

(73) Assignee: Arthrex GmbH, Munchen (DE)

(21) Appl. No.: 15/016,922

(22) Filed: Feb. 5, 2016

Related U.S. Application Data
(63) Continuation of application No. PCT/EP2014/066946, filed on Aug. 6, 2014.

(30) Foreign Application Priority Data
Aug. 6, 2013 (EP) 13179418.2

(51) Int. Cl. A61B 34/20; A61B 17/17

(52) U.S. Cl. CPC A61B 34/20; A61B 17/1703; A61B 2034/2051; A61B 2090/3954

(57) ABSTRACT

A surgical guidance system for orthoscopic drilling is disclosed which comprises a probe, the end of which is suitable for locating on the emergence site of a hole to be drilled through body tissue, a targeter comprising one or more sensors for locating the position of the probe, a drill guide, and a control system in communication with the targeter for determining the position of the probe measured by the targeter and providing a real-time visual representation of the relationship between the direction of the drill guide and the tip of the probe to allow the user to align the drill guide to the tip of the probe. The probe comprises an elongate shaft and one or more, preferably two or more markers located at distant positions along the shaft, and wherein further the markers can be detected by the targeter and are each positioned at locations removed from the tip of the probe.

### PRIORITY CLAIM

This is a continuation of pending International Application No. PCT/EP2014/066946, filed on 6 Aug. 2014, which claims priority from European Application No. 13179418.2, filed on Aug. 6, 2013.

### BACKGROUND OF THE INVENTION

1. Field of the Invention

The invention relates to a guidance system for precisely locating the direction and the depth of a hole to be drilled into a bone or tissue.

2. Description of Relevant Art

Many surgical techniques, for example those of transcoracoid-transclavicular drilling, preferably require the surgeon to make a bore or drill hole through a portion of the patient's body to a desired site, wherein the end of the bore hole is defined within the patient's body but not visible to the surgeon when drilling. If, for example, a surgeon is required to drill through the bone of the patient into the center of a joint, for example to the underside of the joint cartilage, it is desirable for the drill bore to proceed to the desired point within the joint—without the surgeon viewing the end point of the bore from the other side and without damaging the joint. That is, in many situations the surgical procedure is much improved by avoiding a damage of the cartilage, thus meaning that the surgeon uses their skill in locating the bore and stopping the drill at the desired end point within the joint.

A surgical guidance system is disclosed in WO2000/54687. Here, a position measurement device locates a pointer and a drill, both having a plurality of markers. The measured positions are correlated with x-ray images to perform operation planning.

US 2010/0179418 A1 uses an optical tracking system which enables adjustment of a drill versus a pointer to improve aiming of the drill into the direction of the pointer.

US 2001/0036245 A1 discloses a further optical tracking system which increases precision by using data from an x-ray system.

The drawback of these optical tracking systems is the limited accuracy which does not allow to drill holes through a bone into close proximity of a cartilage.

In the U.S. Pat. No. 6,947,788 B2 a magnetic tracking system is disclosed. A trackable catheter includes at least five coils which can be tracked by using an electromagnetic field generated by an electromagnetic transmitter.

### SUMMARY OF THE INVENTION

The embodiments are based on the object of providing a guidance system which allows precise aiming and precise drill depth measurement without using an x-ray imaging system. The guidance system should be comparatively compact and easy to handle. The manufacturing and operating costs should be lower than with comparable guidance systems.

In an embodiment, the system comprises a probe, wherein the probe is preferably utilized for highlighting the end point or a position close to the end point within the patient's body to which the drill bore should extend. As such, the probe preferably has a tip, which may be at the end of a shaft and which may be positioned within a patient's body. Further, the system advantageously comprises a targeter or target module, wherein the targeter is configured to be able to sense the tip of the probe, and thus the end point of a drill line. The system preferably comprises some form of drill guide or drill tube which is included with or integrated into the targeter for providing a guidance to a drill tip when drilling into the patient's body, such that measurements made by the targeter can be used to align the drill guide to the desired end point of the bore to be drilled. The drill guide is defining a drill line. It further may have a reference position for determining the distance of this reference position to the tip of the probe. Preferably, the reference position is at the end and most preferably at the distal end of the drill guide. The tip of the probe may have any shape suitable for pointing to any location or position. It may be a sharp tip, or it may have a ball shape to prevent injuries or damage of tissue.

A control system may be provided for providing a real time visualization of the tip of the probe and the spatial relation to the targeter. In particular, the control system may be provided to highlight the direction in which drilling would proceed through the targeter, and the tip of the probe to ensure that drilling can proceed in a successful manner. The probe preferably comprises an elongate shaft, such that the probe can be inserted into a patient's body. The probe may comprise at least one marker which can be sensed by the targeter. The control system may determine the location of the probe, and in particular the tip of the probe by means of the markers' positions. At least one marker may be located at the tip. Preferably, at least one marker, most preferably, at least two markers are provided at distant positions, further distant from the tip along the shaft. By determining the position of the at least two markers in the shaft by the targeter, the position of the tip may be determined.

Preferably, the guidance system is based on a magnetic tracking system, where the targeter comprises a sensor which may be an electromagnetic or magnetic transmitter to generate a magnetic or electromagnetic field for locating the at least one marker. This differs from the prior art, where a first spatial relation between the probe and a tracker or transmitter is measured. A second spatial relation is measured between the drill and a tracker or transmitter. The relative position between the drill and the probe is then calculated by using these spatial relations. This calculation bears comparatively large errors, as the known tracking systems are comparatively precise in the measurement of positions, but impose significant errors when determining the orientation of an object in space. Preferably, only a distance between the targeter and the tip and/or the at least one marker has to be calculated. It is not required to calculate the distance and direction between two objects. As the measuring distance between the targeter and the tip and/or the at least one marker is smaller than the distance from the tip and the drill to the tracker or transmitter as known from prior art, accuracy of the measurement may further be increased. Due to this smaller distance, the size and weight of the targeter can be reduced to provide a device which can easily be handled. Furthermore, it is preferred, when the drilling direction of the drill corresponds to a preferred direction of the targeter. The targeter may be calibrated to have minimal errors in this preferred direction.

In order to allow the surgeon to properly locate the tip of the probe by means of the targeter, the system may also comprise some means of generating an image for the surgeon such that the tip of the probe, the direction and the end of the drill guide can be readably seen and grasped. This means that the drill guide can be properly aligned to provide a desired drilling direction to the tip of the probe, and the angle of the drill guide can also be shown on the screen to the user to allow for the surgeon to select a desired angle in which to drill.

Preferably, the entire system is devised in such a manner that the visual representation of the targeter, and in particular the direction of the drill guide, and the tip of the probe correlates with the real world directions of the drill guide and probe. The term “real world” is used herein to express the directions or relationships as a user of the system like a surgeon would experience. Actually, the “real world” is related to a user-defined coordinate system or space related to the operating room or the operating table. Alternatively, it may be related to a coordinate system based on the orientation of a surgical drill or tool being used for drilling a hole. If the surgeon is using the guidance system wherein the onscreen or other visual representation of the system matches with the real world directions, movement of the targeter to the left will also mean that the image changes to move the visual representation of the targeter with respect to the probe to the left. It will be understood that this greatly improves the intuitive nature of the system, and leads to great improvement in the accuracy and speed of use of the system in locating the correct position of the drill guide with respect to the probe. Furthermore, in procedures where the angle of the drill guide needs to be known, the system can properly correlate the drill guide angle with the tip of the probe and show this relationship on the visual representation.

A calibration routine or subsection of the control system may be provided, such that the surgeon can properly calibrate the real world orientation of the probe and therefore specify the user-defined coordinate system and store this in the system. By allowing the direction of the probe to be calibrated in use, the surgeon is not restricted in the actual direction in which the probe is positioned into the patient, and greater flexibility of the technique is given.

In order to begin the calibration routine, the control system is preferably adapted to recognize a triggering act or signal. The actual act or signal is not in any way limited, however it is preferable that this act or signal is unlikely to arise during actual surgical procedure, so as to avoid an inadvertent calibration routine rather than proper targeting of the system.

For the triggering act, it may be desirable to have the end of the targeter brought into close proximity with the tip of the probe; for the surgeon to provide a certain predetermined set of motions of the targeter near the probe; to actuate a switch or other element on the probe or targeter; to move the targeter in such a manner that the probe cannot be sensed by the targeter, wherein preferably this could be done for a predetermined length of time.

It is further preferable and possible to provide extra steps in this method, such that after calibration has occurred, the control system can monitor any relative motion between the probe and the targeter and determine this to be a change in the real world orientation of the probe—rather than an actual target procedure. In allowing the continued updating of the real world orientation of the probe, the surgeon does not need to keep the orientation of the probe generally the same after calibration, and further the intuitive nature of the system and accuracy of the surgery will be improved.

A preferable structure to the probe is to comprise markers which can be located by the targeter at a position on the probe which is not going to be positioned within the patient's body during targeting. By keeping the markers which will be sensed by the targeter away from interference of the patient's body, the accuracy and speed of the system will be improved as the patient's body will not cause interference and imaging problems in the system. Whilst there is no real limitation on the markers which are to be sensed and positioned on the probe, these markers could be any one or more of coils, magnets, RFIDs, denser portions of the shaft, other electromagnetically discernible markers. It is also preferable that if multiple markers are provided, each of these markers is different. The difference is in no way limited, however by providing a difference which can be sensed by the targeter, the tip of the probe can be determined by means of the two markers in a quicker and more accurate manner. Most preferably, the targeter is in some way an electromagnetic sensor such that the markers can be readily viewed.

A further modification in the system could be to combine the drill guide or drill tube with the targeter, so that the targeter and drill guide form a combined unit. This has the advantage that the targeter can be used to locate the tip of the probe and then can at the same time properly align the drill guide for performing the desired surgery.

A method for calibration of the above system can also be provided. In particular, the method of use of the above targeter, probe and control system, could allow for some triggering step to redefine any predefined orientation of the probe in the system. Once the triggering step has been registered by the control system, the control system can provide instructions to the user via the real time visual representation, so as to instruct the user how to move one or other of the probe or targeter with respect to the other of the targeter or probe. The system may then monitor the relative motion between the probe and the targeter, and use this to determine the actual real world orientation of the probe in use. Such a method allows for the surgeon to properly program the system to understand the real world direction of the probe, and thus improve the visual representation and visual movement in correlation with the real world movement.

It is preferable that this method be provided with a continued learning phase, wherein after the calibration has occurred the system monitors all movement of the probe with relation to the targeter and uses this to update the real world orientation of the probe. In such a manner, the accuracy of the visual representation can be improved even further thus ensuring that surgery can proceed in a swift and accurate manner.

### BRIEF DESCRIPTION OF THE DRAWINGS

In the following, the invention will be described by way of example, without limitation of the general inventive concept, on examples of embodiment and with reference to the drawings.

FIG. 1 shows a surgical guidance system.

FIG. 2 shows a targeter in detail.

FIG. 3 shows use of the system in a shoulder joint.

FIG. 4 shows use of the system for locating a drilling through a patient's bone.

FIG. 5 shows an example onscreen visual representation showing the guidance of the targeter to the tip of the probe.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

FIG. 1 shows a first embodiment. A surgical guidance system 1 comprises a probe 10 and targeter 20. FIG. 1 shows a general representation of the probe 10 and targeter 20. As mentioned there are certain surgical techniques and procedures, for example Transcoracoid-Transclavicular Drilling or Retrograde Drilling of Osteochondritis Dissecans Lesions, wherein it is preferable to drill through a portion of body tissue to a desired site within the body of a patient from a side where the desired end of the drill site cannot be seen. In these situations, where a surgeon may desire to drill through a bone of a patient to reach the cartilage in a joint, whilst leaving the cartilage generally undamaged by the drilling process, imaging techniques are required to ensure that the drill has both the correct angle and exit point on the bone. Furthermore, it is important to ensure that the drill does not extend too far past the bone into the tissue which is to be protected, and thus in general the precise point as to where the end of the drilled hole should exit the bone, or other body tissue, is desired.

In the system shown in the figures, the targeter 20 may be used to locate the tip of the probe. During a surgical procedure, the surgeon will initially place the probe 10 such that the tip of the probe 12 is located at the desired end point of the hole to be drilled. The positioning of the probe 10 at such a position, and in particular the tip of the probe 12, is less invasive than drilling to this position, or otherwise, and the surgeon can thus ensure that minimal damage to surrounding body tissue ensues from the probe 10 placement. Once the tip of the probe 12 is in the desired location of the body, the targeter 20 can be used to locate the tip of the probe 12, and thus aid the surgeon in drilling to the desired end point and site within the body. Depending on the type of targeting technology, the targeter may instead of locating the tip, locate at least one marker in the probe, where a marker may be located in the shaft of the probe, and calculate thereof the position of the tip.

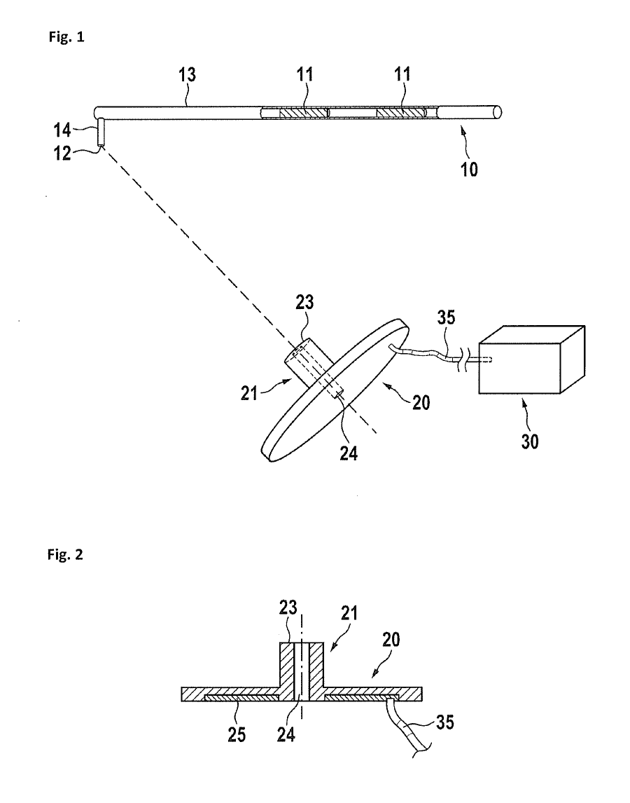

FIG. 2 shows a sectional view of a targeter in detail. The targeter comprises a sensor 25 which may be an electromagnetic or magnetic transmitter to generate a magnetic or electromagnetic field for locating the at least one marker. The sensor may be a coil. Preferably, the sensor is a coil for generating and/or sensing magnetic fields. The sensor may either be used to detect magnetic fields generated by the markers 11 or vice versa to generate magnetic fields which are detected by the markers. The targeter 20 is preferably provided with a drill guide 21, wherein the drill guide 21 can take any form appropriate for ensuring that a drill will proceed in the direction of the drill guide 21. One example would simply be a hole through the targeter 20, or even a hole through the targeter 20 and an extended and/or elongated tube 24 through which a bore passes, to ensure that the drill proceeds in the correct angle. As has been shown in FIG. 1, the targeter 20 and drill guide 21 define a drill line 22, wherein the drill line 22 is the line along which the drill will progress and the bore in the body tissue will be made. As is clear from the description of FIG. 1, once the targeter 20 locates the tip of the probe 12, it is possible to align the drill line 22 with the tip of the probe 12 to ensure that the hole which is to be drilled will proceed in the correct direction and will arrive at the desired end point of the drill bore.

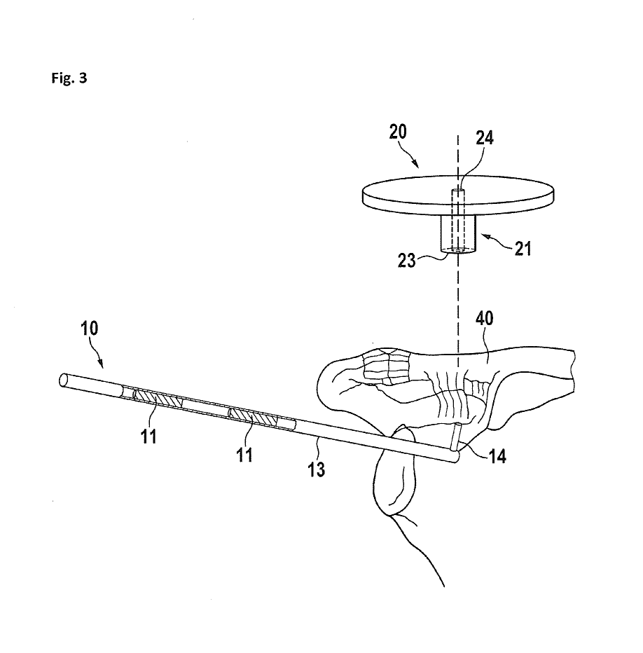

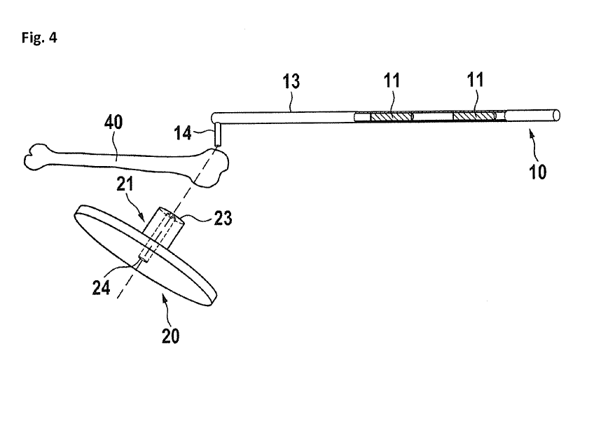

It is also possible by using the system 1 to ensure that the drilled hole does not extend too far. The tip of the probe 12 provides a definite point within the body of the patient, and the targeter 20 can not only guide the angle of the drill line into the direction of the probe 12, but will also be able to determine how far the tip of the probe 12 is from the end of the drill guide 23. This ensures that the bore which is drilled in the bone, or other body tissue, will not extend too far and will end exactly at the tip of the probe 12 or a predetermined distance ahead. As is clear from this technique, the system allows for great flexibility and accuracy in locating a drill hole through body tissue to an end and desired point—without the need for invasive opening of the joint or other visualization techniques, as have been discussed in the prior art as being undesirable. FIGS. 3 and 4 also show the surgical guidance system 1 in use, wherein the probe 10 can be positioned on one side of a bone, and the targeter 20 can be used to align the drill line 22 such that the drilling will proceed in the appropriate direction and will arrive at the tip of the probe 12.

In order to locate the tip of the probe 12, the present system 1 preferably incorporates one or more markers 11 on the probe 10 which can be located by the targeter 20. The nature of the markers 11 is in no way limited to a particular feature or structure. The targeter 20 preferably is an electromagnetic targeter and thus requires only that the position of the one or more markers 11 can be determined by electromagnetic means.

The present case, requires that the minimum invasion of the patient's body be made in order to position the tip of the probe 12 at the desired end site of the drilling hole. The surgeon would usually use an orthoscopic technique to view the joint or other body part to which a hole must be drilled, and further would require or desire to use a small probe 10 for positioning the tip of the probe 12 at the desired end site of the hole. To this end, it is quite clear that the tip of the probe 12—and indeed most of the probe 10 itself, should desirably be of as small a size as possible, thus ensuring that the minimum disruption to the patient's body at the side not being drilled is made. In

Some surgical techniques it is preferred not to interfere with the other side of the drill line as much as possible, and thus the use of large or bulky probes 10 to locate the end of the drilled hole is undesirable.

To this end, and as can be seen in the figures, the probe 10 may be provided with one or more markers 11 at a position away from the tip of the probe 12.

By avoiding positioning the markers 11 which can be sensed by the targeter 20 at the tip of the probe 12, the tip of the probe can be made into a very small and readily positionable point, such that minimum invasion and damage to surrounding tissue occurs. Obviously, however, by providing a very small end to the probe 12 to avoid damage, it is not possible for the targeter 20 to focus and obtain accurate readings of the end 12, as it is also very difficult to put a sufficiently detectable marker 11 at the tip of the very small probe 12.

Furthermore, even if it were possible to put a small marker 11 which can be detected by the targeter 20 at the tip of the probe 12, in many situations the body tissue, and the like, between the targeter 20 and the marker 11 at the tip of the probe 12, would lead to significant interference with the detection of the marker 11, thus reducing the accuracy of the guidance system 1.

As can be seen in the figures, the newly described system 1 proposes the use of one or more markers 11 away from the distal tip of the probe 12. By positioning the markers 11 along the shaft of the probe 13, it is possible to both make the tip of the probe 12 a very small point, and also ensure that the targeter 20 can properly visualize the markers 11 and take an appropriate reading therefrom. It will be noted that in the figures two markers 11 are positioned along the shaft 13 of the probe 10, and that these markers 11 are positioned away from the tip of the probe 12. As can be seen in FIG. 3, the two markers 11 are positioned a sufficient distance away from the tip of the probe 12 such that the two markers 11 are not placed within the body of the patient when the probe 10 is located with the tip of the probe 12 at the desired drilling point. In such a situation, it is clear that the body tissue of the patient does not lie between the targeter 20 and the markers 11, thus ensuring that the position of the markers 11 can be properly detected and monitored by means of the targeter 20. This greatly improves the accuracy of the system, whilst also ensuring that a small tip of the probe 12 can be used to minimize damage to the patient, when in use.

It will be understood that only one marker 11 is strictly necessary in this system, however to improve the accuracy significantly two or more markers 11 can be provided. By positioning the markers 11 along the shaft of the probe 13, it is possible to detect the position of the two or more markers 11. If the two or more markers are measured by the targeter 20, these two points provide a clear direction of the probe 10 in three dimensional space. In order to define a single two dimensional line in a three dimensional space it is necessary to have at least two points, hence the advantage of using two, or more, markers 11 on the probe 10. When the position of the two markers 11 has been determined, it is possible then to determine the precise location of the tip of the probe 12 by extrapolating along the known line. As will be seen from the figures, the probe 10 may be provided with a hook 14 as the tip of the probe 12, wherein the hook 14 can be used in order to also define both the tip of the probe 12 and in some cases also a further extension of the drilling line 22. The positioning of a hook 14 at the tip of the probe 10 is not problematic, as the system 1 can also determine the offset from the markers 11 of the tip of the probe 12.

In this case a control system 30 shown schematically in some form of communication with the targeter 20 or the like in the figures, is provided which is in communication with the targeter 20. The targeter 20 will take measurements and will locate the positions of the one or more markers 11, and can thus then provide this information to the control system 30. The control system 30 then takes the information as to the position of the markers 11, and can calculate the location of the tip of the probe 12. The control system 30 is obviously precalibrated to determine the precise location of the tip of the probe 12 from the measurement of the position of the one or more markers 11, even when the probe 10 is provided with a hook 14. To this end, the measurements taken by the targeter 20 are transferred to the control system 30 this can calculate the relative position of the tip of the probe 12 with regard to the end of the drill guide 23.

Not only is the control system 30 so designed to calculate the relative positions of the tip of the probe 12 and the end of the drill guide 23, but it can also take account of the extension of the drill line 22 with respect to the tip of the probe.

In order to allow the surgeon to properly align the targeter 20 such that the drill line 22 will properly overlap with the tip of the probe 12, it is clear that the targeter 20 will need to be moved in order to align the drill guide 21 appropriately. The control system remains in contact with the targeter 20 and thus obtains real time information of the relative locations of the tip of the probe 12, the end of the drill guide 23 and the drill line 22. By showing the relationship of each of these, for example on a television or computer screen (although there is no specific restriction as to the nature of the representation) the surgeon can obviously move the targeter 20 until the drill line 22 overlaps with the tip of the probe 12. Furthermore, the control system 30 can indicate the distance between the end of the drill guide 23 and the tip of the probe 12, along the drill line 22, such that the surgeon knows exactly how far to drill into the patient to reach the tip of the probe 12. Furthermore, the system is also adapted to be able to highlight the relative angle between the drill line 22 and the tip of the probe 12, and show this on the image. This would allow certain procedures to also ensure that the drill line 22 will not only overlap with the tip of the probe 12, but will also be at a specific angle—which can be advantageous in certain surgical procedures. The system will thus provide a real time image indicating both the tip of the probe 12 as well as the location of the end of the drill guide 23 and the direction of the drill line 22; this will allow the surgeon to move the targeter 20 and align the drill line 22 with the tip of the probe 12.

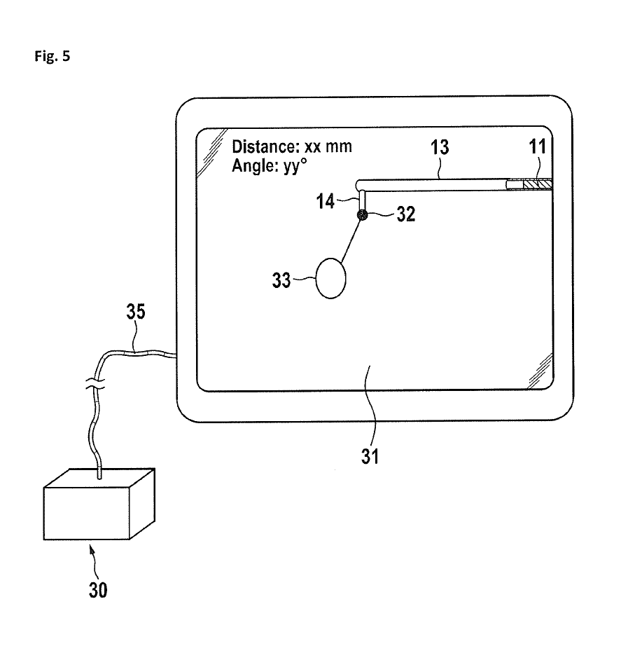

Such a system is schematically shown in FIG. 5, wherein the tip of the probe 12 is highlighted by the dot 32, the circle 33 represents a physical location of the end of the drill guide 23 in relation to the tip of the probe 12, and the line represents the drill line 22. Of course, different representations could be shown, for example the circle 33 shown in FIG. 5 could provide an image showing purely the direction along which the drill line 22 extends, such that movement of the targeter 20 will also move the circle 33, and this need only then be overlapped with the position of the tip of the probe 12 to ensure that the drill will proceed in the correct direction. The nature of the visual representation 31 is not relevant for the teachings of the present disclosure, and any useful graphical, or even numerical, representation given in real time to the surgeon is sufficient for allowing the targeter 20 to be properly aligned with the tip of the probe 12.

As will be appreciated from the above, the guidance system 1 would be greatly improved in usability when the visual representation of the end of the drill guide 23 as well as the drill line 22 and the tip of the probe 12 aligns with the “real world” orientation of the probe 10 and targeter 20. In the current text, the term “real world” will always be used to indicate the physical orientations and positions of all markers of the guidance system 1 in and around the user of the system and patient. The real world orientations are those which the user of the system perceives and in essence lives in, as opposed to the visual representation 31 on the display device of the guidance system 1.

As will be understood, the location of the tip of the probe 12 can be determined from sensing of the one or more markers 11. In particular if multiple markers 11 are provided, then the directional line of the probe 10 can be understood by the control system 30, and the location of the tip of the probe 12 can be derived therefrom. A possible way of determining the actual direction of the probe 10 is to provide the markers 11 with a different nature which can be sensed by the control system 30. For example, if each of the multiple markers 11 were to be different—for example a coil which can be sensed by the EM sensor of the targeter 20, or an RFID tag, or a denser portion of the shaft 13, or the like—it is possible for the actual direction of the probe 10 with respect to the targeter 20 to be determined. Once the control system 30 knows the direction of the probe 10, and in particular the direction of the shaft 13, the position of the tip of the probe 12 can be located with respect to the targeter 20. In particular, the position of the tip of the probe 12 can be determined with respect to the end of the drill guide 23 and the drill line 22.

A problem exists in this system, however, in translating the real world orientation of the probe 10 and targeter 20 onto the visual representation 31 for the surgeon. Unless the control system 30 has knowledge of the actual, real world, orientation of the probe 10 with respect to the user—and not just the targeter 20—the visual representation 31 will not have internal directions which match the real world directions for the user. This therefore means that when the targeter 20 is moved to the left by the user, unless there is a correlation between the real world direction for the user and the visual representation 31 of the control system 30, there is little chance that the movement shown on the screen will match the real world movement of the targeter 20. It is undesirable to have a misalignment between the real world directions and the internal directions of the guidance system 1, as this reduces the intuitive nature of the entire guidance system 1. To this end, it is necessary to determine a relationship between the onscreen directions and the real world directions associated with the probe 10 and targeter 20.

In general, a simple technique would be to always ensure that the probe 10 extends in a known direction to the control system 30, such that once the location of the one or more markers 11, and thus the tip of the probe 12, have been determined, the movement of the targeter 20 is also properly aligned with the image on the screen. Unfortunately, it is very rarely possible to ensure that the probe 10 can be introduced into the drill site and body in a known orientation, consider that this must be done in what is essentially a full three dimensional environment and the precise angle of the probe 10 must align with that stored in the control system 30. As such, the present guidance system 1 proposes some form of calibration to ensure that the real world directions of the probe 10 and targeter 20 are aligned with the visualization directions as determined by the control system 30. By beginning a surgical procedure the user can thus calibrate the known position of the probe 10 with the targeter 20, and ensure that the control system 30 has a clear understanding of the actual physical orientation in the real world of the probe 10 and can thus ensure that relative movements of the targeter 20 will then be shown accurately on the visual representation 31.

A number of mechanisms exist for properly determining the user-defined coordinate system defining the real world orientation of the probe 10, and one will be described here. It is clear, however, that what is required is a possibility of calibrating the physical real world location and orientation of the probe 10 in the control system 30, such that real world movement of the targeter leads to the same movement on the visual representation 31. As such, the disclosed and proposed calibration should not be considered as only the solution to this issue, and the skilled person will readily be able to derive other mechanisms of determining the real world orientation of the probe 10.

As a described example, the user could begin the calibration routine by means of some triggering signal. After the triggering signal is received by the control system 30, the control system 30 can then guide the user of the system through a series of motions of either the probe 10 or targeter 20, to ensure that the location of the probe 10 in the real world can be monitored by the control system 30 such that later relative movements between the targeter 20 and the probe 10 can be coordinated between the real world and the visual representation 31. It is generally easier to provide the probe 10 in a fixed orientation and move the targeter 20 with respect thereto, however the skilled person will realize that the exact opposite will also allow the system 30 to determine the orientation of the probe 10.

The nature of the triggering signal is not really limited to any single technique. It is desirable on the part of the user to avoid having to interact with anything other than the probe 10 and targeter 20, and thus a preferred technique is generally one which requires the user not to have to interact with the control system 30 directly or physically. For example, the end of the drill guide 23 could be brought into contact or very near contact with the tip of the probe 12, this can be registered by the control system 30 as a triggering signal which will then lead the control system 30 to begin a calibration routine. Further possibilities are that a known motion of the targeter 20 could be used in relation to the probe 10 in order to begin the calibration routine. Such a motion of the targeter 20 would be advantageously rather large and unlikely to occur in any surgical situation, so as to avoid inadvertent starting of the calibration routine during an actual surgical procedure. Further, the triggering signal could be the physical removal of the targeter 20 away from the probe 10, such that the field of view of the targeter 20 no longer comprises the probe 10 at all, and maintaining the targeter 20 away from the probe 10 for a sufficiently long time to ensure resetting of the system. As is clear from the above, any form of motion between the probe and targeter 20 can be used to start the calibration routine, and thus the system is not really limited to any of the possibilities.

Once the calibration routine has started, the simplest mechanism of determining the user-defined coordinate system corresponding to the real world orientation of the probe 10 by the control system 30, is to lead the user of the system through a series of defined motions of the targeter 20 in the real world. For example, moving the targeter 20 to the left; followed by to the right; followed by up; followed by down, will lead the control system 30 to fully understand the physical orientation of the probe 10 in the real world, and thus the probe 10 orientation can be stored and the visual representation 31 between the motion of the targeter 20 with respect to the probe 10 can be coordinated for easy use by the surgeon. Other motions of the targeter 20 can also be used to indicate the location of the probe 10, and in fact the probe 10 can be moved instead of moving the targeter 20. The actual mechanism by which the control system 30 determines the orientation of the probe 10 is, as stressed a second time, not limited, and it is the provision of the calibration routine in order for the control system 30 to learn the real world orientation of the probe 10 that is important.

Once the orientation of the probe 10 is known to the control system 30, movement of the targeter 20 with respect to the probe 10 will then match between the real world and the visual representation 31, thus improving the intuitive nature of the system for the user. Given that any surgical procedure needs to be achieved with the greatest accuracy in the shortest amount of time, to avoid unwanted infection to the patient for one, this improvement to the intuitiveness is greatly appreciated. Further, by means of this simple calibration routine, accuracy of the surgery will also be increased as the surgeon can focus on ensuring that the drill line 22 and length of the drilled hole will properly match such that the tip of the probe 12 is met.

It will be understood from the above that after the calibration routine has been followed, the orientation of the probe 10 in the user-defined coordinate system corresponding to the real world should probably not change too much from that of the targeter 20. In order to allow the surgeon to generally determine the real world orientation of the probe 10 and store this in the control system 30, and then have freedom to position the probe 10 at the desired surgical site, a preferable further step to the calibration method is to allow for the system to monitor the movement of the probe 10 and update the stored probe 10 orientation. As can be understood, once the orientation of the probe 10 is determined at the end of the calibration routine, the targeter 20 could be maintained in its known orientation and monitor the motion of the probe 10 as this is placed into the desired surgical site. After either a predetermined length of time, or length of inactivity of the motion of the probe 10 with respect to the targeter 20, or indeed any other predetermined further signal, the control system 30 could stop updating the orientation of the probe 10 and then allow for the relative movement between the tip of the probe 12 and the targeter 20 to be understood as motion of the targeter 20 to align the drill line 22 to the tip of the probe 12. That is, after a certain amount of learning time, the control system 30 assumes that the probe 10 is in location such that the tip of the probe 12 now marks the desired end point of the drill, such that the motion of the targeter 20 is then treated as a targeting motion, rather than a calibration motion. In this manner, even motion of the probe 10 after calibration will be updated as the orientation of the probe in the system, further increasing the accuracy of the system 1.

A further advantage of the second probe 10 orientation storage, is that should the surgeon need to dramatically change the orientation of the probe 10 in the real world as a result of trying to get to the site at the end of the drill hole, it will not be necessary to go through the calibration routine a second time. Again this improves the interaction of the surgeon with the display and greatly improves the accuracy of using the guidance system 1. Furthermore, the speed of using the system 1 is increased which again reduces the length of a surgical procedure which is always desirable. In the above description and figures it is clear that the system is shown with a combined targeter 20 and drill guide 21, however this is for convenience only—at all points the skilled person will appreciate that the targeter 20 could be separate from the drill guide 21. Indeed, in some surgical techniques it is actually preferable for the drill guide 21 to be a separate item and not part of the targeter in such cases the surgeon could locate the separate drill guide 21 in the desired orientation and position—based on the measurements made by the targeter 20 for locating the tip of the probe 12. An assistant could hold the targeter 20 behind the separate drill guide 21 along the same line as the drill line 22 of the drill guide 21, so as to allow alignment of the drill guide 21 with the tip of the probe 12. Another possibility would be to provide the separate drill guide 21 with markers, in an analogous manner to those positioned on the probe 10, such that the control system 30 could also measure the line location and direction of the drill guide 21 in relation to the tip of the probe 12, so as to allow the targeter 20 to be generally located and sensing the surgical site, wherein the control system 30 takes the measurements from both the probe 10 and the separate drill guide 21 and provide an appropriate image for the surgeon on the visual representation 31.

As will be understood above, a number of features and aspects relating to the control system 30, probe 10 and targeter 20 have been described. From this description the skilled person understands that many markers can be derived as both individual markers of the system and markers in combination with other features. No clear description of marker combinations should be understood from the above disclosure over and above what would be logical in order to allow for determination of the end of the drill hole, and appropriate calibration of the control system 30 to the real world direction. To this end, the above should be considered as a description of aspects and concepts, which when logical can be combined to generate an appropriately functioning system. The claims attached to this disclosure provide a description of the key aspects of the disclosure.

It will be appreciated to those skilled in the art having the benefit of this disclosure that this invention is believed to provide a surgical guidance system. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A surgical guidance system for orthoscopic drilling comprising:
a probe having a shaft with at least one marker and a tip for locating a position,
a targeter comprising at least one sensor for locating the position of the at least one marker,
a drill guide defining a drill line,
a control system in communication with the targeter for determining the position of the tip based on information provided by the targeter and providing a real-time visual representation of the relationship between the direction of the drill guide and the tip of the probe to allow a user to align the drill guide to the tip of the probe, wherein
the targeter includes the drill guide and
the surgical guidance system is based on a magnetic tracking system, where the at least one sensor comprises a magnetic transmitter to generate a magnetic for locating the at least one marker.

2. The system of claim 1, wherein the probe comprises an elongate shaft and at least two markers located at distant positions along the shaft further being distant from the tip of the probe.

3. The system of claim 1, wherein the control system is adapted to determine the distance between the targeter and the tip of the probe and/or the angle between the drill line and the tip of the probe.

4. The system of claim 1, wherein the control system is adapted to provide a visual representation indicating the location of the tip of the probe, and the drill line of the drill guide, in relation to the tip of the probe.

5. The system of claim 4, wherein the control system is adapted to provide the visual representation with respect to the orientation of the probe, the drill guide and the targeter in a user-defined coordinate system, such that a movement of the targeter and/or the drill guide leads to a corresponding movement of the image showing the direction of the drill guide on the screen, so as to improve the intuitive use of the instrument for the user.

6. The system of claim 1, wherein the control system is adapted to perform a calibration of the user-defined coordinate system, thus allowing the probe to be oriented in any orientation with respect to the targeter.

7. The system of claim 1, wherein the control system is adapted to perform a calibration routine to determine the user-defined coordinate system after a triggering act has been performed by the user.

8. The system of claim 7, wherein the triggering act is at least one of:
bringing an end of the drill guide into close proximity or contact with the tip of the probe,
performing a predefined set of motions of the targeter near the probe,
actuating a switch on the targeter or a switch on the probe,
moving the targeter so that the probe is out of its field of view for more than a predetermined time.

9. The system of claim 7, wherein for a period of time after the calibration routine has been successfully performed, the control system is adapted to monitor the relative movement of the probe with respect to the targeter and to update the calibration direction should orientation changes of the probe occur.

10. The system of claim 1, wherein the distance between the at least one marker and the tip of the probe is larger than the body part into which the tip of the probe will be positioned.

11. The system of claim 1, wherein the at least one marker is at least one of coils, magnets, RFIDs, denser portions of the shaft, or any other electromagnetically discernible markers and preferably the targeter comprises an electromagnetic sensor for imaging the one or more markers on the shaft.

12. The system of claim 1, wherein the targeter comprises an electromagnetic sensor for locating the at least one marker.

13. A method for calibrating the surgical guidance system according to claim 1, comprising:
performing a triggering step for triggering a calibration routine,
providing visible instructions to the user via the visual representation for moving either one of the targeter or probe with respect to the other of the probe or targeter,
monitoring the relative position of the probe and targeter by the control system,
determining a user-defined coordinate system from the relative movement between the probe and the targeter and storing this user-defined coordinate system.

14. The method according to claim 13, wherein once the control system has determined and stored the user-defined coordinate system, the control system provides real time images wherein the motion of the targeter and drill guide in the user-defined coordinate system are properly calibrated with the visual representation, such that movement of the targeter and/or drill guide in the user-defined coordinate system provides the same movement on the visual representation.

15. The method according to claim 13, wherein after the step of determining the user-defined coordinate system and storing this, the method continues by:
monitoring the relative motion between the targeter and probe, wherein changes to the orientation of the probe are registered and after a set time and/or a period of time where no change in orientation of the probe occurs the new orientation of the probe is determined to be the actual user-defined coordinate system and is stored.
